(19) United States
(12) Patent Application Publication
(10) Pub. No.: US 2006/0257400 A1
(43) Pub. Date: Nov. 16, 2006

(54) COMBINATION THERAPY

(75) Inventor: Joseph Fargnoli, Pipersville, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company, Princeton, NJ (US)

(21) Appl. No.: 11/382,742

(22) Filed: May 11, 2006

Related U.S. Application Data
(60) Provisional application No. 60/680,692, filed on May 13, 2005.

(51) Int. Cl. A61K 39/395; A61K 31/53

(52) U.S. Cl. 424/143.1; 514/243

(57) ABSTRACT

This invention relates to a therapeutic combination of anti-cancer compounds which comprises a) a VEGFR-2 inhibitor, and b) a substance that binds to the epidermal growth factor receptor (EGFR) and blocks the ability of epidermal growth factor (EGF) to initiate receptor activities which results in tumor growth inhibition, and optionally at least one pharmaceutically acceptable carrier for simultaneous, separate or sequential use.

### RELATED APPLICATION

This application claims priority benefit under Title 35 § 119(e) of U.S. provisional Application No. 60/680,692, filed on May 13, 2005, the contents of which are herein incorporated by reference.

### FIELD OF THE INVENTION

This invention relates to a combination of anti-cancer compounds which comprises a) a Vascular Endothelial Growth Factor-2 (VEGFR-2) Inhibitor, and b) an epidermal growth factor receptor (EGFR) antibody, and optionally at least one pharmaceutically acceptable carrier for simultaneous, separate or sequential use.

### BACKGROUND OF THE INVENTION

The VEGFR-2 receptor is a tyrosine protein kinase receptor that drives angiogenesis, a process critical for tumor growth and metastasis. Angiogenesis is a complex and highly regulated process. A substantial number of growth factors and cytokines have been identified in recent years that activate and maintain angiogenesis throughout tumorgenesis. There are three VEGF receptors that are implicated in angiogenesis, however, VEGFR-2 is the most highly validated in angiogenesis among these three receptors because it has been implicated in multiple steps, including endothelial cell proliferation, survival, migration and differentiation as well as vascular permeability.

EGFR antibodies can be selected from chimerized, humanized, fully human, and single chain antibodies derived from the murine antibody 225 described in U.S. Pat. No. 4,943,533 to Mendelsohn et al. The EGFR antibody can be, for example, cetuximab which is marketed as Erbitux™ by ImClone Systems, Inc. and Bristol-Myers Squibb Company. The EGFR antibody can also be selected from the antibodies described in U.S. Pat. No. 6,235,883 to Jakobovits et al., U.S. Pat. No. 5,558,864 to Bendi et al., and U.S. Pat. No. 5,891,996 to Mateo de Acosta del Rio et al.

### SUMMARY OF THE INVENTION

This invention relates to a combination of anti-cancer compounds which comprises a) a VEGFR-2 Inhibitor, and b) an EGFR antibody, and optionally at least one pharmaceutically acceptable carrier for simultaneous, separate or sequential use.

In particular, it has been found that the VEGFR-2 inhibitor compounds, when administered essentially simultaneously with an epidermal growth factor receptor antibody, exhibited better than additive antitumor activity.

More particularly, it has been found that VEGFR-2 inhibitor compounds, when administered essentially simultaneously with the epidermal growth factor receptor antibody, cetuximab, exhibited better than additive antitumor activity in a predictive mouse model.

### BRIEF DESCRIPTION OF THE DRAWINGS

FIG. 1. Anti-tumor Activity of Compound I when Combined with Cetuximab.

### DETAILED DESCRIPTION OF THE INVENTION

It has been found that certain VEGFR-2 inhibitor compounds, when administered either simultaneously or sequentially with an EGFR antibody, exhibit antitumor activity in a predictive mouse model. The invention also relates to methods of treating cancer and other proliferative diseases using the therapeutic combination of compounds.

A particular VEGFR-2 inhibitor compound of the formula (hereinafter referred to as Compound I) 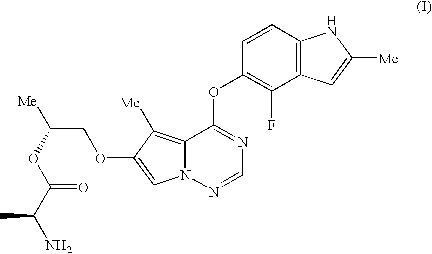
or a pharmaceutically acceptable salt, solvate, ester or isomer thereof, is used in the combination and methods of the invention. This compound and other VEGFR-2 inhibitor compounds, which may show similar properties, and their preparation is disclosed in U.S. Pat. No. 6,869,952, the disclosure of which is incorporated herein by reference. Crystal forms of compound I are disclosed and claimed in U.S. Provisional Application No. 60/721,021, filed Sep. 27, 2005, the disclosure of which is incorporated herein by reference.

The EGFR antibodies can be selected from chimerized, humanized, fully human, and single chain antibodies derived from the murine antibody 225 described in U.S. Pat. No. 4,943,533 to Mendelsohn et al. The EGFR antibody can be, for example, cetuximab which is marketed as Erbitux™ by ImClone Systems, Inc. and Bristol-Myers Squibb Company. The EGFR antibody may also be selected from the antibodies described in U.S. Pat. No. 6,235,883 to Jakobovits et al., U.S. Pat. No. 5,558,864 to Bendi et al., and U.S. Pat. No. 5,891,996 to Mateo de Acosta del Rio et al.

The EGFR monoclonal antibody, Erbitux® (cetuximab) was found to provide the therapeutically synergistic antitumor activity in vivo when combined with the oral taxane.

The nature of proliferative diseases like solid tumor diseases is multifactorial. Under certain circumstances, drugs with different mechanisms of action may be combined. However, just considering any combination of drugs having different modes of action does not necessarily lead to combinations with advantageous effects. In fact, drugs within the same class may not all have the same effect when used in combination.

It has been surprisingly found that the combination of Compound I plus EGFR monoclonal antibody, cetuximab, provided antitumor activity in a predictive mouse model. It was found that there was a delay in tumor growth when both agents were combined that was greater than the sum of each treatment alone.

It can be shown by established test models and in particular those models described herein that the combination of the invention results in better activity compared to the effects observed with the single combination partners. The pharmacological activity of the combination of the invention may be further demonstrated in a clinical study as well as in the procedure described herein.

In one embodiment of the invention, each patient receives an EGFR antibody, such as cetuximab, on a weekly or other clinically useful schedule, at dose levels typically used for the particular EGFR antibody involved. In the specific instance of cetuximab, that might include an initial dose of 400 mg/m² followed thereafter by 250 mg/m² weekly, or a regimen of similar dose levels adjusted for optimal use in the combination setting. The VEGFR-2 inhibitor compound, Compound I, could be administered on any clinically useful schedule, including, but not limited to, daily, twice weekly, weekly or every other week. Specifically, for daily administration, typical dosages of Compound I might range from 2 to 1000 mg/m², adjusted as the clinician saw fit, to accommodate any developing patient needs.

The following are definitions of terms that may be used in the present specification. The initial definition provided for a group or term herein applies to that group or term throughout the present specification individually or as part of another group, unless otherwise indicated.

The VEGFR-2 inhibitor compounds of the invention may form salts which are also within the scope of this invention. Pharmaceutically acceptable (i.e. non-toxic, physiologically acceptable) salts are preferred, although other salts are also useful, e.g., in isolating or purifying the compounds of this invention.

The VEGFR-2 inhibitor compounds of the invention may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The VEGFR-2 inhibitor compounds of the invention may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention includes all the possible stereoisomers and their mixtures. Particularly preferred are the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The combination of the invention is useful in the treatment of a variety of cancers, including (but not limited to) the following:
carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma;
hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkitt's lymphoma;
hematopoietic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastoma, glioma and schwannomas; and
other tumors, including melanoma, seminoma, teratocarcinoma, osteosarcoma, xenoderoma pigmentosum, keratoctanthoma, thyroid follicular cancer and Kaposi's sarcoma.

### EXAMPLES

### Materials And Methods

Compounds. Compound I was synthesized by Bristol-Myers Squibb (BMS) chemists. Cetuximab (also known as Erbitux®) was a gift of Imclone Systems, Inc. The antibody was dissolved in phosphate buffered saline for i.p. injection to mice. Compound I was administered to mice in a volume of 100 mg/kg of body weight based on the average weight of the mice in each group at the time of treatment. Cetuximab was administered in 0.25 ml on a per mouse basis.

Animals. Athymic (“nude”) mice, 5-6 weeks of age, purchased from Harlan Sprague Dawley (Indianapolis, Ind.), were quarantined for ˜2 weeks before their use for tumor propagation and drug efficacy testing. They were fed food and water ad libitum. All studies involving these animals were conducted in accordance with NIH (Bethesda, Md.) and Bristol Myers-Squibb animal care and use guidelines.

Tumors. Human L2987 lung carcinomas were maintained in nude mice by serial s.c. passage. All efficacy testing involved tumors implanted s.c. in nude mice. Treatment was initiated when tumors had become well established at between 100-200 mm³.

### Antitumor Testing

Compound I was administered in combination with Erbitux and compared to either agent dosed alone. Each compound was dosed on its optimal preclinical schedule and dose level. This included a daily regimen for Compound I at 100 mg/Kg while Erbitux was dosed at 1 mg per mouse every three days for a total delivery of 5 doses.

When Compound I and cetuximab were both administered to mice, they were given essentially simultaneously, with no attempt at any particular sequence applied.

### Results

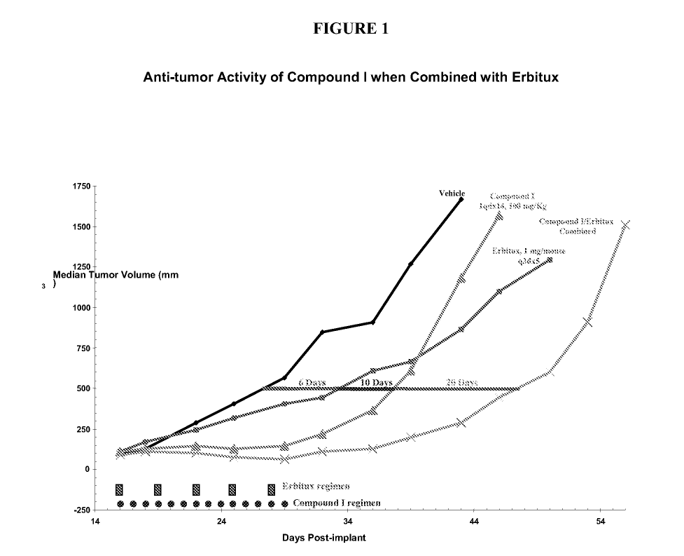

As shown in FIG. 1, Erbitux when dosed alone delayed tumor growth by 6 days when compared to a tumor size of 500 mm³ in the vehicle treated control group while Compound I delayed tumor growth to this same target size for ten days. When both agents were combined, a 20 day delay in reaching this same target size was observed, which is greater than the sum of each treatment alone. Based on this result, combination therapy with these agents is feasible and results in potentiation of tumor growth delay. Further studies can be planned to determine if this combination is synergistic by using lower than optimal doses of each compound.

Despite advances in the past decade, patients with NSCLC and other tumors are in need of more effective therapeutic interventions. The preclinical data presented here, demonstrating some additional delay in tumor when the VEGFR-2 inhibitor, Compound I and EGFR antibody, cetuximab, were combined suggest an approach that ought to be evaluated clinically in appropriate indications.

We claim:

1. A pharmaceutical combination of anti-cancer compounds which comprises
a) a VEGFR-2 inhibitor, and
b) an epidermal growth factor receptor antibody,
in which the active ingredients are present in each case in free form or as a pharmaceutically acceptable salt, solvate or ester.

2. The combination according to claim 1 wherein the VEGFR-2 inhibitor is a compound of the formula 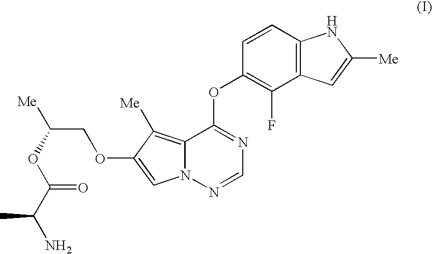
or a pharmaceutically acceptable salt, solvate, ester or isomer thereof.

3. The combination according to claim 1 wherein the epidermal growth factor receptor antibody is chimerized, humanized, fully human, or a single chain antibody.

4. The combination according to claim 3 wherein the epidermal growth factor receptor antibody is cetuximab.

5. The combination according to claim 1 wherein the VEGFR-2 inhibitor is a compound of the formula 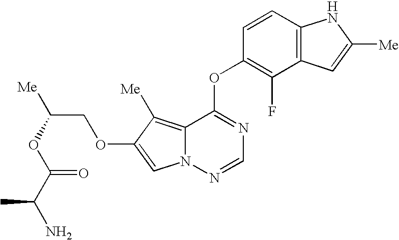
or a pharmaceutically acceptable salt, solvate, ester or isomer thereof; and
the epidermal growth factor receptor antibody is cetuximab.

6. The combination according to claim 5 wherein the VEGFR-2 inhibitor is administered at a dose of about 2 to 1000 mg/m² every 1 to 14 days for 1 or more administrations.

7. The combination according to claim 5 wherein cetuximab is administered at a dose of 4 to 400 mg/m². i.v. every 1 to 14 days for 1 or more administrations.

8. The combination according to claim 5 wherein the two compounds are administered essentially simultaneously.

9. A method for the treatment of cancer which comprises administering a
a) a VEGFR-2 inhibitor, and
b) an epidermal growth factor receptor antibody,
in which the active ingredients are present in each case in free form or as a pharmaceutically acceptable salt, solvate or ester.

10. The method according to claim 9 wherein the VEGFR-2 inhibitor is a compound of the formula 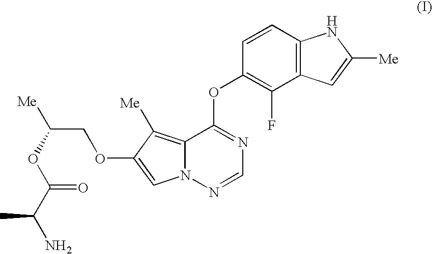
or a pharmaceutically acceptable salt, solvate, ester or isomer thereof.

11. The method according to claim 9 wherein the epidermal growth factor receptor antibody is chimerized, humanized, fully human, or a single chain antibody.

12. The method according to claim 11 wherein the epidermal growth factor receptor antibody is cetuximab.

13. The method according to claim 9 wherein the VEGFR-2 inhibitor is a compound of the formula 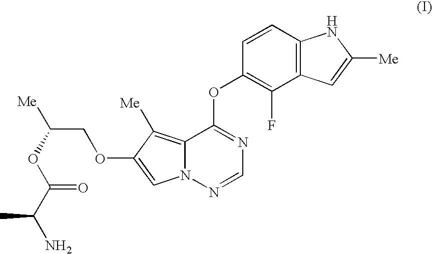
or a pharmaceutically acceptable salt, solvate, ester or isomer thereof.

14. The method according to claim 9 wherein the two compounds are administered essentially simultaneously.

15. The method according to claim 9 wherein the cancer treated is selected from colorectal cancer, breast cancer, gastric cancer, ovarian cancer, non-small cell lung cancer and cancers of the head and neck.
